(19) United States
(12) Patent Application Publication
(10) Pub. No.: US 2015/0070019 A1
(43) Pub. Date: Mar. 12, 2015

(54) CONTROL APPARATUS

(71) Applicant: SEIKO EPSON CORPORATION, Tokyo (JP)

(72) Inventors: Daisuke KAWAKAMI, Matsumoto-shi (JP); Shinichi MIYAZAKI, Suwa-shi (JP); Hiroyuki YOSHINO, Suwa-shi (JP)

(21) Appl. No.: 14/483,434

(22) Filed: Sep. 11, 2014

(30) Foreign Application Priority Data
Sep. 11, 2013 (JP) 2013-187984

(51) Int. Cl. G01R 31/327; A61B 17/3203; G06F 19/00

(52) U.S. Cl. CPC G01R 31/3278; G06F 19/3406; A61B 17/3203; A61B 2017/00185
USPC 324/418

(57) ABSTRACT

A control apparatus controls a detachable electric instrument. The control apparatus includes a drive signal output unit configured to output a drive signal to the electric instrument; a relay provided in a sending route of the drive signal; a switching unit configured to output a switching signal that switches the relay between ON and OFF; an inspection signal output unit configured to output an inspection signal that inspects the relay to the sending route in a state in which the electric instrument is not connected; and a blocking determining unit configured to determine whether the inspection signal is blocked by the relay in a state in which the relay is turned OFF by the switching signal.

This application claims the benefit of Japanese Patent Application No. 2013-187984, filed on Sep. 11, 2013. The content of the aforementioned patent application is incorporated by reference herein in its entirety.

### BACKGROUND

1. Technical Field

The present invention relates to control.

2. Related Art

As a liquid injection apparatus used as a medical instrument, there is a known configuration including a hand piece configured to inject liquid and a control apparatus configured to control an operation of the handpiece. The control apparatus is configured to cause the handpiece to execute and stop injection of liquid in accordance with an input from a user (For Example, JP-A-2012-047071).

The configuration of the related art described above is superior configuration which achieves incision and resection without using a knife. The inventors studied about an inspection procedure when starting the usage of the apparatus as described above and have gotten to the invention given below. In addition, a reduction in size, a reduction in cost, resource saving, ease of manufacture, and an improvement of user-friendliness are also required. The inventors have made an attempt to solve these challenges as well.

### SUMMARY

An advantage of some aspects of the invention is to solve at least one of the problems described above, and the invention can be implemented as the following forms.

(1) An aspect of the invention provides a control apparatus configured to control a detachable electric instrument is provided. The control apparatus includes: a drive signal output unit configured to output a drive signal to the electric instrument; a relay provided on a sending route of the drive signal; a switching unit configured to output a switching signal that switches the relay between ON and OFF; an inspection signal output unit configured to output an inspection signal that inspects the relay to the sending route in a state in which the electric instrument is not connected; and a blocking determining unit configured to determine whether the inspection signal is blocked by the relay in a state in which the relay is turned OFF by the switching signal. In this configuration, whether a function of blocking the signal is normally operated before connecting the electric instrument is determined, and hence unintentional input of the drive signal to the electric instrument after the electric instrument has connected is avoided.

(2) This aspect of the invention is directed to the aspect described above, wherein the electric instrument is a medical instrument, and the inspection signal output unit outputs the inspection signal in a state in which the medical instrument is not connected. In this configuration, unintentional input of the drive signal into the medical instrument is avoided.

(3) This aspect of the invention is directed to the aspect described above, wherein the control apparatus includes: a first monitoring unit configured to output a signal indicating a result of monitoring of a voltage upstream of the relay; and a second monitoring unit configured to output a signal indicating a result of monitoring of a voltage downstream of the relay; and the first and second monitoring units output the signals indicating the result of monitoring, and the blocking determining unit is configured to compare a signal output from the first monitoring unit and a signal output from the second monitoring unit. In this aspect, whether the inspection signal is blocked by the relay can be determined in a simple configuration.

(4) This aspect of the invention is directed to the aspect described above, wherein the control apparatus includes: an opening determining unit configured to determine whether the inspection signal is passed through the relay in a state in which the relay is turned ON by the switching signal. In this aspect, input of the drive signal into the electric instrument is confirmed.

(5) This aspect of the invention is directed to the aspect described above, wherein the opening determining unit is configured to compare a signal output from the first monitoring unit and a signal output from the second monitoring unit. In this aspect, whether the drive signal into the electric instrument is input into the electric instrument is determined in a simple configuration.

(6) This aspect of the invention is directed to the aspect described above, wherein the signal output by the first monitoring unit indicates whether the voltage input into the relay is not smaller than a threshold value, the signal output by the second monitoring unit indicates whether the voltage output from the relay is not smaller than the threshold value, the threshold value is set to be a value smaller than the maximum voltage generated by the inspection signal in the case where the inspection signal is output, and the maximum voltage generated by the inspection signal is smaller than the maximum voltage generated by the drive signal. In this aspect, the inspection may be executed by using the inspection signal at a lower voltage than the drive signal.

(7) This aspect of the invention is directed to the aspect described above, wherein the threshold value is set to be a value larger than the maximum voltage generated by the drive signal in the case where the drive signal is output. In this aspect, output of the signal more than necessary at the time of output of the drive signal is avoided.

(8) Another aspect of the invention provides a control apparatus configured to control the detachable medical instrument is provided. The control apparatus includes: a drive signal output unit configured to output a drive signal to the medical instrument; a relay provided in a sending route of the drive signal; a switching unit configured to output a switching signal that switches the relay between ON and OFF; and an inspection signal output unit configured to output an inspection signal to the relay. The control apparatus may be configured to detect the inspection signal passed through the relay when the inspection signal output unit outputs the inspection signal to the relay in a state in which the relay is turned OFF by the switching signal. In this configuration, the control apparatus can detect if the inspection signal passes through the relay in a state in which the relay provided in the sending route of the drive signal is OFF before connecting the medical instrument. Therefore, unintentional output of the drive signal into the medical instrument is detected.

The invention may be implemented in various forms other than those described above. For example, the invention may be implemented in forms such as an inspection method, a program for implementing this method, a storage medium having these programs stored therein. Alternatively, the invention may be implemented in forms of a liquid injection apparatus, a liquid injection method, a medical instrument, and a method of surgical operation provided with the above-described control apparatus.

### BRIEF DESCRIPTION OF THE DRAWINGS

The invention will be described with reference to the accompanying drawings, wherein like numbers reference like elements.

FIG. 1 is a configuration drawing of a liquid injection apparatus.

FIG. 2 is a block diagram illustrating an internal configuration of a control apparatus.

FIG. 3 is a flowchart showing a relay inspection process.

FIG. 4 is a flowchart showing an opening inspection process.

FIGS. 5A to 5D are graphs showing a waveform in the opening inspection process.

FIG. 6 is a flowchart showing a connection inspection process.

FIGS. 7A to 7D are graphs showing a waveform in the connection inspection process.

FIGS. 8A to 8D are graphs showing respective waveform at the time of injection of liquid.

### DESCRIPTION OF EXEMPLARY EMBODIMENTS

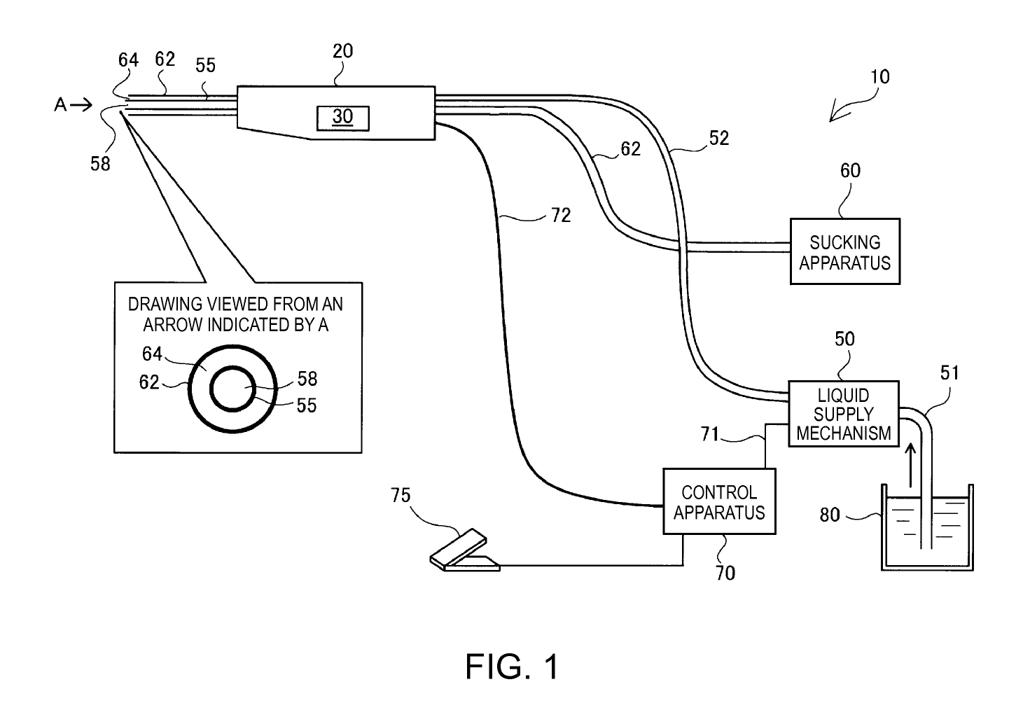

A first Embodiment will be described. FIG. 1 illustrates a configuration of a liquid injection apparatus 10. The liquid injection apparatus 10 is a medical instrument used in a medical organization, and has a function to incise and resect an affected area by injecting liquid toward the affected area.

The liquid injection apparatus 10 includes a handpiece 20, a liquid supply mechanism 50, a sucking apparatus 60, a control apparatus 70, and a liquid container 80. The liquid supply mechanism 50 and the liquid container 80 are connected to each other by a connecting tube 51. The liquid supply mechanism 50 and the handpiece 20 are connected to each other by a liquid supply flow channel 52. The connecting tube 51 and the liquid supply flow channel 52 are formed of a resin. The connecting tube 51 and the liquid supply flow channel 52 may be formed of a material other than the resin (a metal, for example).

The liquid container 80 stores normal saline solution. The liquid may be pure water or drug solution instead of the normal saline solution. The liquid supply mechanism 50 supplies liquid sucked from the liquid container 80 via the connecting tube 51 to the handpiece 20 via the liquid supply flow channel 52 by driving a pump integrated therein.

The handpiece 20 is an instrument that a user of the liquid injection apparatus 10 operates by holding in his or her hand. The user performs incision or resection of an affected area by injecting the liquid injected intermittently from the handpiece 20 onto the affected area.

The sucking apparatus 60 is configured to suck liquid or resected tissue around the injection port 58. The sucking apparatus 60 and the handpiece 20 are connected to each other by a sucking flow channel 62. The sucking apparatus 60 sucks an interior of the sucking flow channel 62 constantly while the switch for driving the sucking apparatus 60 is ON. The sucking flow channel 62 penetrates through an interior of the handpiece 20, and opening in the vicinity of a distal end of an injection tube 55.

The sucking flow channel 62 lays over the injection tube 55 extending from a distal end of the handpiece 20. Therefore, as illustrated in a drawing viewed in a direction indicated by A in FIG. 1, a wall of the injection tube 55 and a wall of the sucking flow channel 62 form concentric cylinders. A flow channel in which a sucked material sucked from a suction port 64 which corresponds to the distal end of the sucking flow channel 62 flows is defined between an outer wall of the injection tube 55 and an inner wall of the sucking flow channel 62. The sucked material is sucked to the sucking apparatus 60 via the sucking flow channel 62.

The handpiece 20 is a disposable product, and is replaced by a new product at every surgical operation. The liquid supply flow channel 52, the sucking flow channel 62 and a signal cable 72 (hereinafter, these three are referred to collectively as “cables”) are fixed to the handpiece 20, and is replaced together with the handpiece 20. When a new handpiece 20 is used, the handpiece 20 to which cables are connected, and the cables are connected to respective connecting points.

When the user turns a foot switch 75 ON in a state in which the cables are connected, the control apparatus 70 sends a drive signal to a pulsation generating unit 30 integrated in the handpiece 20 via the signal cable 72. The pulsation generating unit 30 generates pulses in a pressure of supplied liquid upon input of the drive signal. With this pulsation, an intermittent injection of the above-described liquid is realized. The pulsation generating unit 30 executes generation of pulses by using expansion and contraction of a piezoelectric element integrated therein. The drive signal is for expanding and contracting the piezoelectric element.

However, liquid is injected in the case where the foot switch 75 is turned ON as described above is only in the case where the control apparatus 70 is set to a permission mode. The control apparatus 70 sets itself to either the permission mode or a prohibition mode. Even when the foot switch 75 is turned ON in the case of the prohibition mode, the control apparatus 70 does not drive the pulsation generating unit 30 and the liquid supply mechanism 50. Therefore, in the case of the prohibition mode, no liquid is injected.

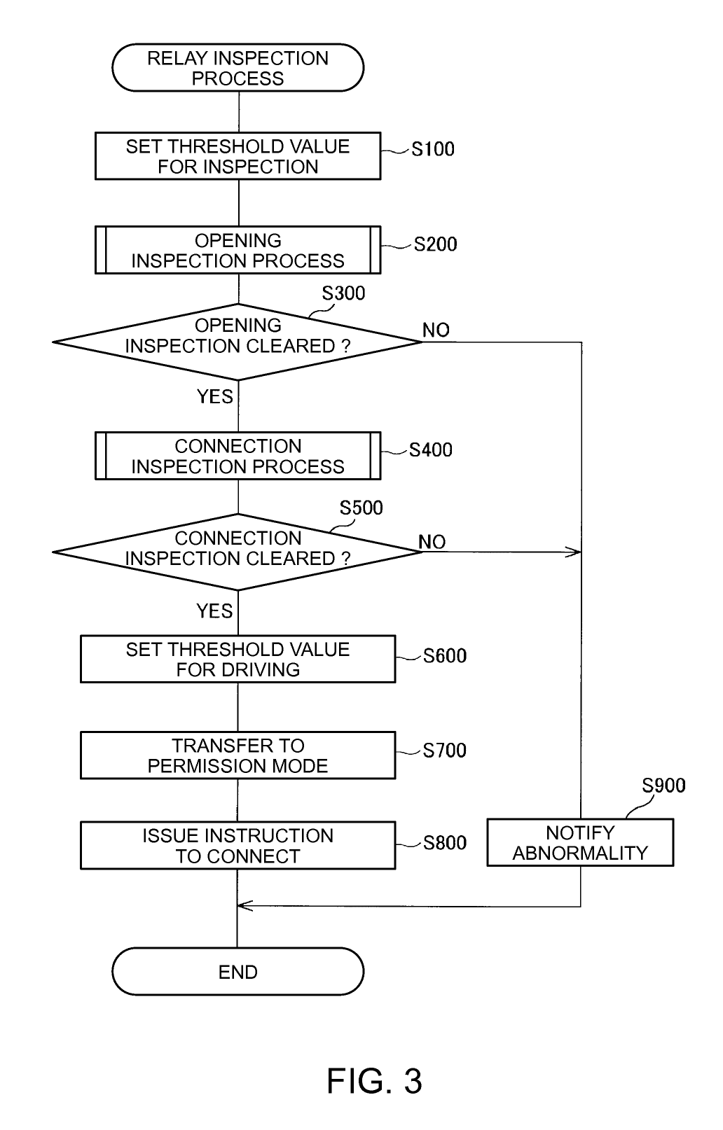

A default mode of the control apparatus 70 is the prohibition mode. Transfer to the permission mode is executed only when a relay inspection process (described later in conjunction with FIG. 3.) is executed before the connection of the signal cable 72 and the inspection is cleared. The permission mode is maintained until the signal cable 72 is connected after the transfer to the permission mode and then the signal cable 72 is disconnected.

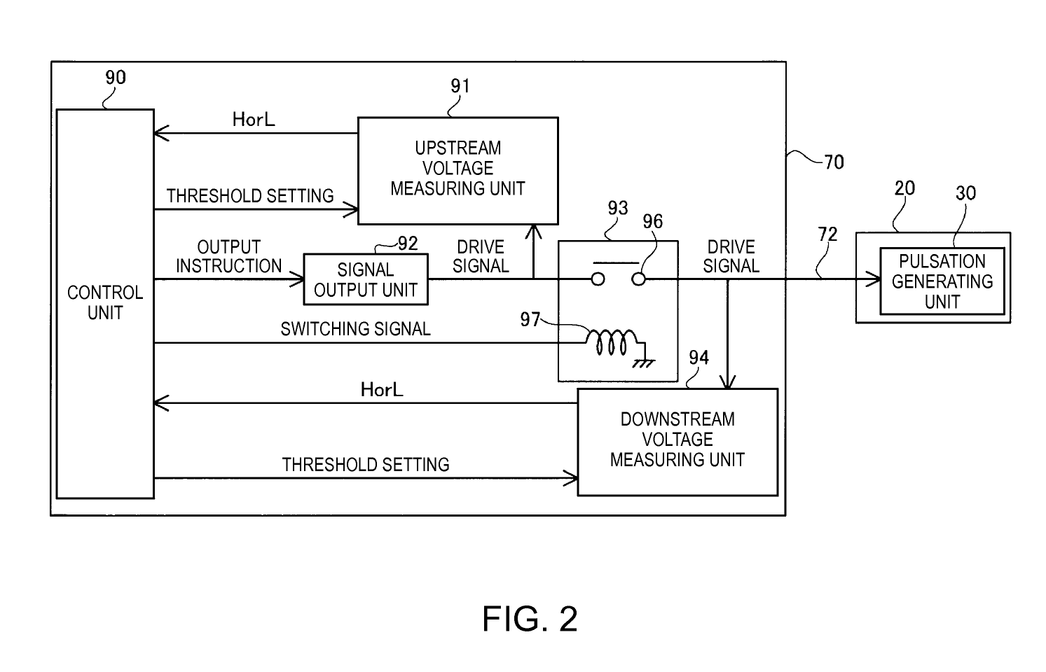

FIG. 2 is a block diagram illustrating an internal configuration of the control apparatus 70, and illustrating a state in which the control apparatus 70 and the handpiece 20 are connected via the signal cable 72. The control apparatus 70 includes a control unit 90, an upstream voltage measuring unit 91, a signal output unit 92, a relay 93, and a downstream voltage measuring unit 94. The relay 93 is an electromagnetic relay, and includes a contact point 96 and a solenoid 97.

The control unit 90 is composed of a microcomputer, and issues an instruction to output a drive signal to the signal output unit 92. The signal output unit 92 outputs the drive signal upon reception of the instruction. The drive signal output from the signal output unit 92 is input to the relay 93 and the upstream voltage measuring unit 91. In a state in which the contact point 96 is closed (hereinafter, referred to as “relay 93 is ON”), the drive signal passes through the relay 93 and is input to the pulsation generating unit 30 via the signal cable 72.

The control unit 90 switches the relay 93 between ON and OFF (a state in which the contact point 96 is opened) by inputting a switching signal to the solenoid 97 of the relay 93. In the permission mode descried above, the relay 93 is maintained at ON and in the prohibition mode, the relay 93 is maintained at OFF. The contact point 96 is a normally-opened contact point. Therefore, the relay 93 is ON in the state in which the switching signal is input, and is OFF in the state in which the switching signal is not input.

The upstream voltage measuring unit 91 inputs an upstream measuring signal to the control unit 90. The upstream measuring signal is a digital signal to be set to a value H if the voltage value upstream of the relay 93 is not smaller than a threshold value, and to a value L if smaller than the threshold value. The expression “upstream of the relay 93” means a portion between the signal output unit 92 and the relay 93. The threshold value is a variable value determined by the control unit 90.

The downstream voltage measuring unit 94 inputs a downstream measuring signal to the control unit 90. The downstream measuring signal is a digital signal to be set to a value H if the voltage value downstream of the relay 93 (between the relay 93 and the pulsation generating unit 30) is not smaller than a threshold value, and to a value L if smaller than the threshold value. The threshold value is a variable value determined by the control unit 90.

FIG. 3 is a flowchart showing a relay inspection process. The relay inspection process is executed by the control unit 90 upon a pressing operation of a setup switch provided on the control apparatus 70 in a state in which the handpiece 20 is not connected to the control apparatus 70 via the signal cable 72. As will be described later, when the inspection in this process is cleared, the mode is transferred form the prohibition mode to the permission mode.

First of all, the threshold value to be used by the upstream voltage measuring unit 91 and the downstream voltage measuring unit 94 is set to a threshold value Th1 for inspection (Step S100). Subsequently, an opening inspection process is executed (Step S200).

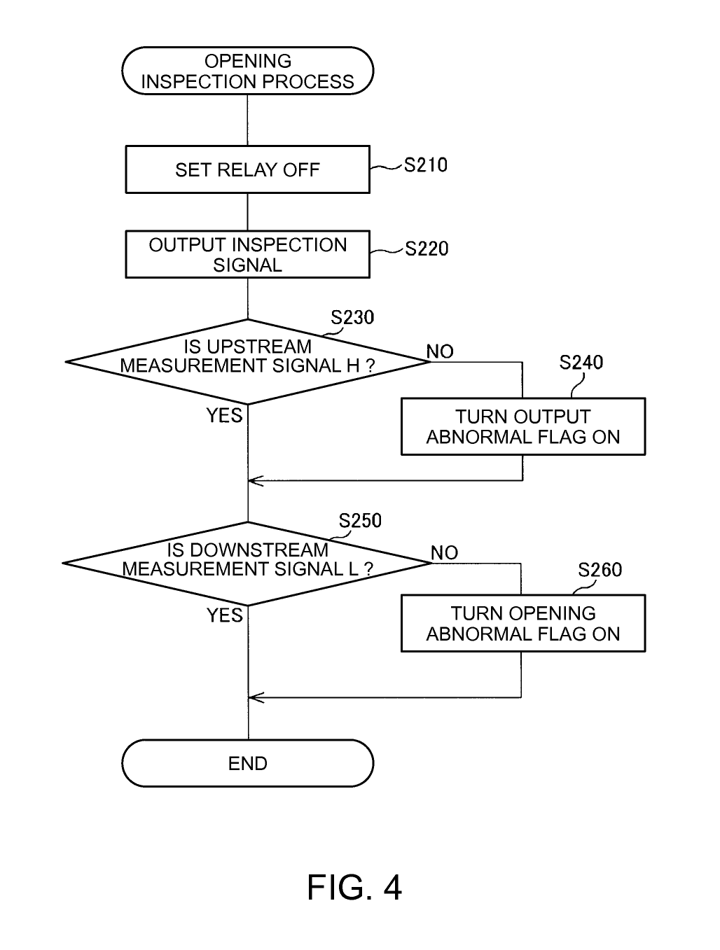

FIG. 4 is a flowchart showing the opening inspection process. First of all, the relay 93 is set to OFF (Step S210). Subsequently, an inspection signal is output to signal output unit 92 (Step S220). The output inspection signal is input to the upstream voltage measuring unit 91. Subsequently, whether the upstream measuring signal reaches the value H in a predetermined period is determined (Step S230).

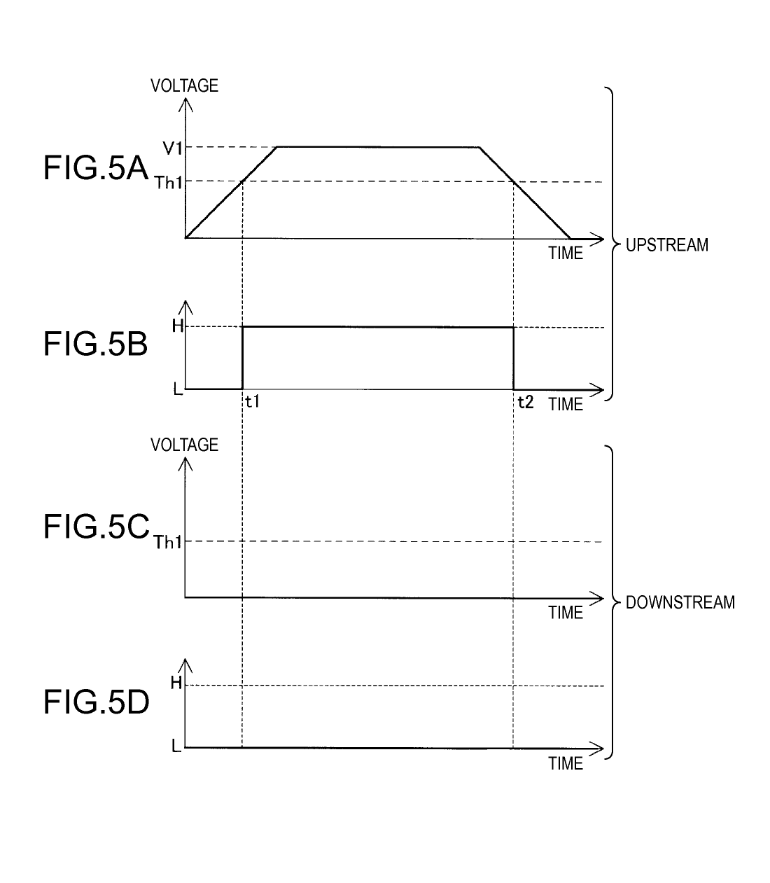

A graph of FIG. 5A shows a waveform of the inspection signal. In other words, the graph (A) shows a voltage value obtained by the upstream voltage measuring unit 91 in the case where the inspection signal is output. As shown in the graph of FIG. 5A, the voltage value of the inspection signal is linearly increased from zero to a voltage V1 when the output of the inspection signal starts, and after having reached the voltage V1, is maintained at the voltage V1 for a predetermined period. After an elapse of the predetermined period, the voltage value is linearly decreased to zero. The voltage V1 is larger than the threshold value Th1 as shown in the graph of FIG. 5A. Maintaining the voltage at V1 for a predetermined period means maintaining the voltage V1 within a predetermined voltage range.

A graph of FIG. 5B is a graph showing an upstream measuring signal in the case where the inspection signal is output. As shown in the graphs of FIGS. 5A and 5B, the voltage value of the inspection signal is increased to be values not smaller than the threshold value Th1 between a clock time t1 to a clock time t2. Therefore, the upstream measuring signal becomes the value H in this period.

The above-described Step S230 is a step for confirming whether the output by the signal output unit 92 is normally executed on the basis of the fact that the upstream measuring signal is at the value H during the predetermined period (between the clock time t1 and the clock time t2) and at the value L before the clock time t1 and after the clock time t2 as described above.

In the case where the upstream measuring signal is not the value H in the predetermined period (No in Step S230), an output abnormal flag is turned on (Step S240). As a condition to determine YES in Step S230, the period during which the upstream measuring signal becomes the value H needs not to be strictly between the clock time t1 and the clock time t2, and a time difference within a predetermined range is allowed.

Whether the downstream measuring signal was the value L in the case where the upstream measuring signal becomes the value H in the period between the clock time t1 and the clock time t2 (YES in Step S230) or after the output abnormal flag has been turned on is determined (Step S250).

A graph of FIG. 5C shows a voltage value obtained by the downstream voltage measuring unit 94 in the case where the inspection signal is output in the opening inspection process. A graph of FIG. 5D is a graph showing a downstream measuring signal in the case where the inspection signal is output in the opening inspection process. In the opening inspection process, since the relay 93 is set to OFF, the inspection signal is not input to the downstream voltage measuring unit 94.

Step S250 described above is a step for determining whether the relay 93 blocks the drive signal normally on the basis of the fact that the upstream measuring signal becomes the value H but the downstream measuring signal is the value L. In the case where the downstream measuring signal becomes the value H even though it is for a short time (No in Step S250), an opening abnormal flag is turned on (Step S260) in order to show the result of inspection that the relay 93 cannot be set to OFF, and the opening inspection process is terminated. Examples of reasons why the relay 93 cannot be set to OFF include welding at the contact point 96. The process described thus far corresponds to the process as a blocking determining unit.

In contrast, in the case where the downstream measuring signal is maintained at the value L (YES in Step S250), the opening inspection process is terminated without turning the opening abnormal flag on.

Subsequently, whether the opening inspection is cleared is determined (Step S300). Specifically, in the case where neither the output abnormal flag nor the opening abnormal flag is turned on, it is determined that the opening inspection is cleared. In the case where the opening inspection is cleared (YES in Step S300), a connection inspection process is executed (Step S400).

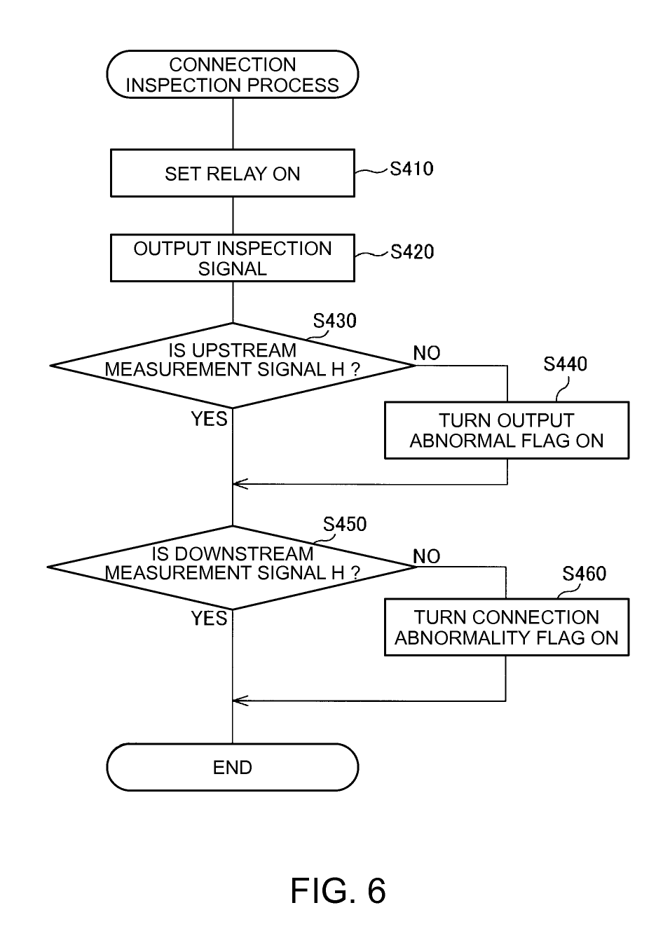

FIG. 6 is a flowchart illustrating the connection inspection process. First of all, the relay 93 is set to ON (Step S410). Subsequently, the inspection signal is output to the signal output unit 92 (Step S420). The inspection signal output here is a signal having the same waveform as that used in the opening inspection process. The processes of Step S210 and Step S410 in the control unit 90 correspond to the process of the switching unit.

Subsequently, whether the upstream measuring signal becomes the value H is determined (Step S430). In the case where the upstream measuring signal is not the value H (NO in Step S430), an output abnormal flag is turned on (Step S440). The methods and the objects of the steps S430 and S440 are the same as those of Steps S230 and S240 in the opening inspection process.

In the case where the upstream measuring signal becomes the value H (YES in Step S430) or after the output abnormal flag has been turned on, whether the downstream measuring signal becomes the value H is determined (Step S450).

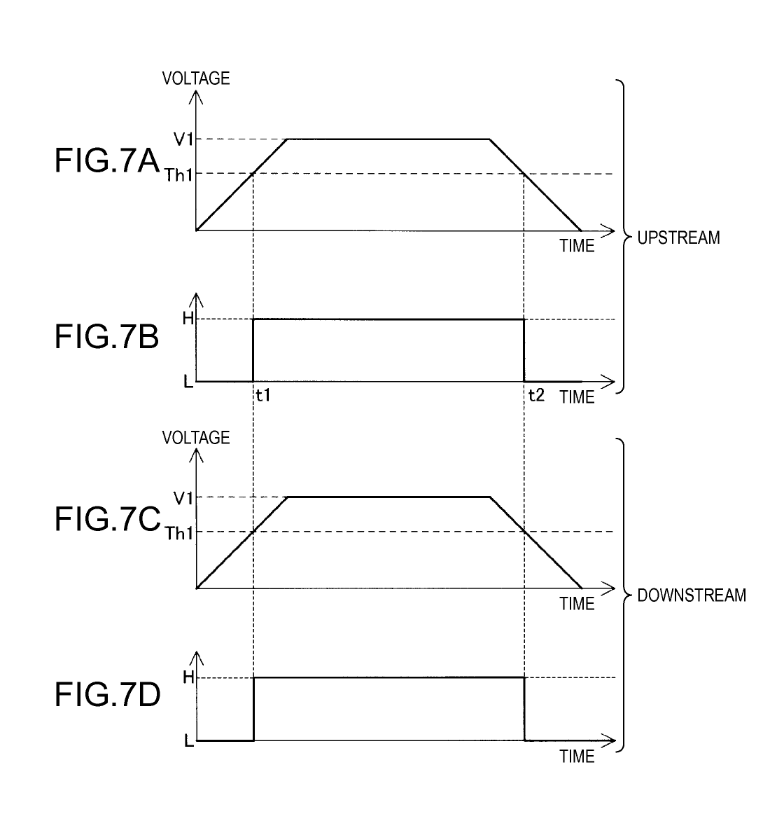

A graph of FIG. 7A is a graph showing a waveform of the inspection signal, and a graph of FIG. 7B is a graph showing an upstream measuring signal in the case where the inspection signal is output. Since the graphs of FIGS. 7A and 7B are the same as the graphs of FIGS. 5A and 5B, detailed description will be omitted.

A graph of FIG. 7C shows a voltage value obtained by the downstream voltage measuring unit 94 in the case where the inspection signal is output in the connection inspection process. A graph of FIG. 7D is a graph showing the downstream measuring signal in the case where the inspection signal is output in the connection inspection process.

In the connection inspection process, since the relay 93 is set to ON, the inspection signal is input to the downstream voltage measuring unit 94. In this manner, since the upstream voltage measuring unit 91 and the downstream voltage measuring unit 94 receive an input of the same signal and are set to have the same threshold value Th1, the upstream measuring signal and the downstream measuring signal become the value H in the same period.

Step S450 described above is a step for determining whether the relay 93 allows the drive signal to pass therethrough normally on the basis of the fact that the downstream measuring signal has the same waveform as the upstream measuring signal. In the case where the downstream measuring signal does not become the value H (No in Step S450), a connection abnormal flag is turned on (Step S460) in order to show the result of inspection that the relay 93 cannot be set to ON, and the connection inspection process is terminated.

In contrast, in the case where the downstream measuring signal becomes the value H in the same manner as the upstream measuring signal (YES in Step S450), the connection inspection process is terminated without turning the connection abnormal flag on.

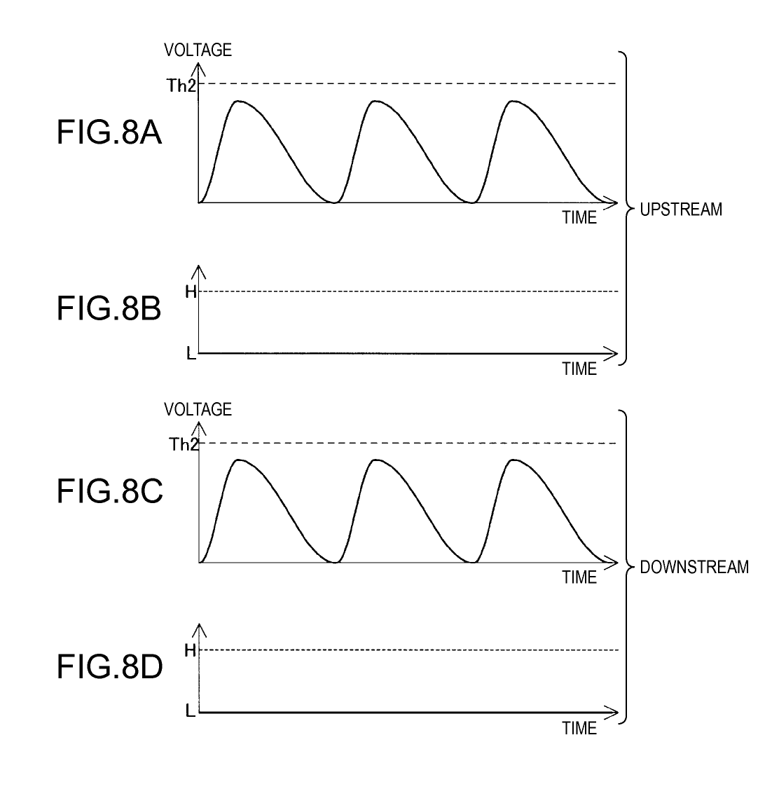

Subsequently, whether the connection inspection is cleared is determined (Step S500). Specifically, in the case where both of the output abnormal flag and the connection abnormal flag is not turned on, it is determined that the connection inspection is cleared. In the case where the connection inspection is cleared (YES in Step S500), the threshold value is set to a threshold value Th2 (see FIGS. 8A to 8D) (Step S600), and the mode is transferred to the permission mode (Step S700). Finally an instruction is issued to the user to connect the cables (Step S800), and the relay inspection process is terminated. The instruction of connection is achieved by displaying a massage such as “Connect the cables.” on a display provided on the control apparatus 70.

In contrast, in the case where the opening inspection is not cleared (NO in Step S300) or in the case where the connection inspection is not cleared (NO in Step S500), the abnormality is notified to the user (Step S900), the relay inspection process is terminated while maintaining the prohibition mode. Notification of abnormality is executed by displaying a message such as “Send the unit to repair” on the display provided on the control apparatus 70 or outputting a buzzer sound.

FIGS. 8A to 8D show graphs in the case where the drive signal is output in the permission mode. Vertical axes of graphs of FIGS. 8A to 8D represent a voltage value obtained by the upstream voltage measuring unit 91, the upstream measuring signal, a voltage value obtained by the downstream voltage measuring unit 94, and the downstream measuring signal, respectively. Lateral axes represent time.

In the case where the drive signal is output, as shown in the graph of FIG. 8A, the upstream voltage measuring unit 91 receives an input of the drive signal. Furthermore, since the mode is the permission mode, the relay 93 is set to ON, and hence the downstream voltage measuring unit 94 receives an input of the drive signal as illustrated in the graph of FIG. 8C.

As illustrated in the graphs of FIGS. 8A and 8C, the voltage value of the drive signal increases and decreases cyclically. Depending on the cyclical increase and decrease of the voltage value, the piezoelectric element integrated in the pulsation generating unit 30 expands and contracts cyclically, whereby intermittent injection of the liquid is achieved.

As described above, in the case of the permission mode, the threshold value Th2 is set to the upstream voltage measuring unit 91 and the downstream voltage measuring unit 94. In other words, when the voltage value of the drive signal reaches Th1 in the permission mode, the upstream voltage measuring unit 91 and the downstream voltage measuring unit 94 output the value H. The threshold value Th2 is a value higher than the maximum value of the normal drive signal. Therefore, when the drive signal is output normally, the upstream measuring signal and the downstream measuring signal are always the value L. In this manner, while the drive signal is output, these signals are maintained at the value L to avoid the application of a load to the control unit 90.

According to the embodiment, whether the control apparatus 70 can block and output the drive signal normally may be inspected before connecting the handpiece 20 to the control apparatus 70. Furthermore, in the inspection, the upstream voltage measuring unit 91 and the downstream voltage measuring unit 94 output digital signals indicating the result of comparison with respect to the threshold values, and hence the control unit 90 is capable of determining whether the apparatus is normal or abnormal.

The invention is not limited to the embodiments, examples, and modifications in this specification and may be implemented in various configurations without departing the scope of the invention. For example, technical characteristics in the embodiments, the examples, and the modifications corresponding to the technical characteristics in the respective embodiments in the respective modes described in the paragraph of the summary may be replaced or combined as needed in order to solve part or entire problem described above or in order to achieve part or entire part of the above-described advantages. The technical characteristics may be eliminated as needed unless otherwise specified to be essential in the specification. For example, the followings are exemplified.

The contact point of the relay may be a normally-closed contact point, or may be a type in which the opening and closing of the contact point is switched every time when the current flows.

The output of the switching signal may be changed depending on a change of the operation of the relay.

The type of the relay may be a solid state relay or a program relay. The solid state relay is not provided with a mechanical contact point. However, the “contact point” of this application is not limited to the mechanical contact point, but also includes a configuration for achieving ON and OFF in the solid state relay.

The connection inspection do not have to be executed.

The control unit may be determined on the basis of an analogue signal on at least one of the opening inspection and the connection inspection. In this case, the control unit may obtain the voltage values at upstream and downstream of the relay without using the upstream voltage measuring unit and the downstream voltage measuring unit.

The inspection signal and the drive signal may be monitored by separate hardware.

The waveform of the inspection signal may be changed. For example, the waveform of the inspection signal may be a triangle wave in which the voltage value changes so as to straddle the threshold value.

The relay inspection process may be executed after the cables are connected. In this case, the inspection may be performed without departing from a range of voltage value which does not cause a problem even though the drive signal is unintentionally input to the pulsation generating unit.

The handpiece and the cables may not be fixed. For example, the cables may be fixed to the control apparatus, the liquid supply mechanism, and the sucking apparatus.

At least two of the drive signal output unit configured to output the drive signal, the switching unit configured to output the switching signal for switching ON and OFF of the relay, and the blocking determining unit configured to determine whether the inspection signal is blocked by the contact point may be processed by one CPU. By processing one CPU, a reduction in size or a reduction in cost of the control apparatus may be achieved. The process described above may be performed dispersedly with a plurality of CPUs. By the dispersing process, a load on a single CPU is reduced, so that the high-speed process may be executed.

There may be provided with a plurality of the relays, or the plurality of the relays may be connected in series. If there is the plurality of the relays, even though the current of one of the relays cannot be blocked, the current can be blocked with a relay which can block the current.

One or the plurality of the relay configured to input the inspection signal may be provided. By providing one relay for inputting the inspection signal, the circuit configuration may be simplified, and the ON and OFF can be switched by the relay which can block the signal and the medical instrument can be used while notifying the fact that one of relay cannot block the current. Therefore, the control apparatus may be applied to emergent therapies. By providing the plurality of the relays configured to input the inspection signal, the control apparatus having higher reliability may be provided.

The liquid injection apparatus has been described as the handpiece. However, the liquid injection apparatus may be a liquid injection apparatus used in an endoscope. The liquid injection apparatus does not have to be a disposable product, and may be replaced with a new product at every surgical operation.

It is also possible to output the inspection signal for inspecting the relay to the sending route in a state after the liquid injection apparatus and the control apparatus have been connected and the liquid injection apparatus has used and the liquid injection apparatus is not connected, and determine whether the inspection signal is blocked by the contact point in a state in which the relay is turned OFF. Consequently, whether the function to block the signal works normally may be determined before performing the control apparatus for the next time, so that the determination may be omitted and the liquid injection apparatus can be used in an early stage. In addition, abnormality of the control apparatus can be detected in an early stage.

The liquid injection apparatus may be used in applications other than the medical instrument.

For example, the liquid injection apparatus may be used in a cleaning apparatus configured to clean the stain by injected liquid.

The liquid injection apparatus may be used in a drawing apparatus configured to draw a line with injected liquid.

The system of the liquid injection may be that using a laser beam. The injection system using the laser beam utilizes a pressure variation generated by irradiating liquid with a laser beam intermittently and gasifies the liquid.

The medical instrument of the invention is not limited to the liquid injection apparatus and may be applied to an electric surgical knife, or an ultrasonic surgical knife.

What is claimed is:

1. A control apparatus configured to control a detachable electric instrument comprising:
a drive signal output unit configured to output a drive signal to the electric instrument;
a relay provided in a sending route of the drive signal;
a switching unit configured to output a switching signal that switches the relay between ON and OFF;
an inspection signal output unit configured to output an inspection signal that inspects the relay to the sending route in a state in which the electric instrument is not connected; and
a blocking determining unit configured to determine whether the inspection signal is blocked by the relay in a state in which the relay is turned OFF by the switching signal.

2. The control apparatus according to claim 1, wherein
the electric instrument is a medical instrument, and
the inspection signal output unit outputs the inspection signal in a state in which the medical instrument is not connected.

3. The control apparatus according to claim 1, further comprising:
a first monitoring unit configured to output a signal indicating a result of monitoring of a voltage upstream of the relay; and
a second monitoring unit configured to output a signal indicating a result of monitoring of a voltage downstream of the relay, wherein
the first and second monitoring units output the signals indicating the result of monitoring, and
the blocking determining unit is configured to compare a signal output from the first monitoring unit and a signal output from the second monitoring unit.

4. The control apparatus according to claim 3, comprising:
an opening determining unit configured to determine whether the inspection signal is passed through the relay in a state in which the relay is turned ON by the switching signal.

5. The control apparatus according to claim 4, wherein
the opening determining unit is configured to compare a signal output from the first monitoring unit and a signal output from the second monitoring unit.

6. The control apparatus according to claim 3, wherein:
the signal output by the first monitoring unit indicates whether the voltage input into the relay is not smaller than a threshold value,
the signal output by the second monitoring unit indicates whether the voltage output from the relay is not smaller than the threshold value,
the threshold value is set to be a value smaller than the maximum voltage generated by the inspection signal in the case where the inspection signal is output, and
the maximum voltage generated by the inspection signal is smaller than the maximum voltage generated by the drive signal.

7. The control apparatus according to claim 6, comprising:
the threshold value is set to be a value larger than the maximum voltage generated by the drive signal in the case where the drive signal is output.

8. A control apparatus configured to control a detachable medical instrument comprising:
a drive signal output unit configured to output a drive signal to the medical instrument;
a relay provided in a sending route of the drive signal;
a switching unit configured to output a switching signal that switches the relay between ON and OFF;
an inspection signal output unit configured to output an inspection signal to the relay, wherein
the inspection signal passed through the relay is detected when the inspection signal output unit outputs the inspection signal to the relay in a state in which the relay is turned OFF by the switching signal.
